(19) United States
(12) Patent Application Publication
(10) Pub. No.: US 2026/0027329 A1
(43) Pub. Date: Jan. 29, 2026

(54) MAGNETIC MEDICAL DEVICE COMPONENTS AND METHODS OF USE THEREOF

(71) Applicant: Northwestern University, Evanston, IL (US)

(72) Inventors: Ahsun RIAZ, Evanston, IL (US); Ali HUSNAIN, Evanston, IL (US)

(21) Appl. No.: 19/280,957

(22) Filed: Jul. 25, 2025

Related U.S. Application Data
(60) Provisional application No. 63/675,619, filed on Jul. 25, 2024.

(51) Int. Cl. A61M 25/01; A61F 2/01; A61F 2/95; A61M 25/09

(52) U.S. Cl. CPC A61M 25/0127; A61F 2/011; A61F 2/95; A61M 25/09041; A61F 2002/9528; A61F 2210/009; A61M 2205/0272

(57) ABSTRACT

Provided herein are medical devices with magnetic components that allow for locating, repositioning, and/or retrieving medical devices within a subject. In particular, a magnetically-tipped wire and/or an external magnet are provided, for example, as components of system with other medical devices and/or for use in medical procedures, such as in the field of interventional radiology (IR).

### CROSS-REFERENCE

This application claims the benefit of U.S. Provisional Patent Application No. 63/675,619, filed Jul. 25, 2025; which is incorporated by reference herein in its entirety.

### FIELD

Provided herein are medical devices with magnetic components that allow for locating, repositioning, and/or retrieving medical devices within a subject. In particular, a magnetically tipped wire, a magnetic sheath/catheter, and/or an external magnet are provided, for example, as components of system with other medical devices and/or for use in medical procedures, such as in the field of interventional radiology (IR).

### BACKGROUND

Interventional radiology, gastroenterology, and urology procedures often involve the placement and retrieval of implanted devices such as stents, inferior vena cava (IVC) filters, and other medical implants. The successful retrieval of these devices is critical for patient care and can present significant challenges for clinicians.

Current methods for retrieving implanted devices typically rely on mechanical engagement using snares, forceps, or similar tools. However, these methods can be problematic, particularly when dealing with devices that have shifted position or become misaligned within the body. For example, an IVC filter that has tilted can be difficult to engage with conventional retrieval tools, potentially leading to prolonged procedure times and increased risk to the patient. Additionally, in cases where a device such as a stent is the only structure crossing a stricture, removal of the device results in loss of access across the stricture. This necessitates re-establishing access, which can be challenging and time-consuming, and may not always be successful.

Furthermore, as medical procedures trend towards increased automation and robotics, there is a growing need for technologies that can reduce the level of skill required for complex alignment tasks. Current methods often rely heavily on the operator's expertise and can involve significant guesswork, which is not ideal for automated systems. The limitations of existing retrieval technologies can lead to several negative outcomes, including failure to locate or successfully retrieve implanted devices, prolonged procedure times, increased risk of complications, loss of critical access across strictures, and difficulty in aligning components for retrieval or further intervention.

There is a clear need for innovative approaches that can improve the alignment, engagement, and retrieval of implanted medical devices. Such innovations could potentially reduce procedure times, decrease the skill level required for complex interventions, and improve overall patient outcomes in the fields of interventional radiology, gastroenterology, and urology.

### SUMMARY

Provided herein are medical devices with magnetic components that allow for locating, repositioning, and/or retrieving medical devices within a subject. In particular, a magnetically tipped wire, a magnetic sheath/catheter, and/or an external magnet are provided, for example, as components of system with other medical devices and/or for use in medical procedures, such as in the field of interventional radiology (IR).

In some embodiments, provided herein are medical device systems comprising a guidewire comprising an elongate flexible body and at least one magnetic element disposed at a distal portion of the body, a catheter comprising a lumen configured to receive the guidewire and at least one magnetic element disposed at a distal portion of the catheter, and an external magnetic device configured to generate a magnetic field detectable within a body of a subject.

In some embodiments, the at least one magnetic element of the guidewire comprises a plurality of cylindrical magnets aligned in series.

In some embodiments, the guidewire comprises a non-magnetic material selected from the group consisting of nitinol, stainless steel, and combinations thereof. In some embodiments, the guidewire has a length between 50 cm and 300 cm and a diameter between 0.014 inches and 0.038 inches.

In some embodiments, the at least one magnetic element of the sheath/catheter comprises a plurality of cylindrical magnets.

In some embodiments, the catheter has an outer diameter between 3 Fr and 16 Fr.

In some embodiments, the external magnetic device comprises a rod-shaped permanent magnet having a length between 10 cm and 50 cm and a diameter between 1 cm and 5 cm.

In some embodiments, the system further comprises an implantable medical device having a ferromagnetic component configured to interact with the at least one magnetic element of the guidewire.

In some embodiments, provided herein are methods for manipulating an implanted medical device, the method comprising advancing a catheter through a lumen of a subject to a location proximate to the implanted medical device, the catheter comprising at least one magnetic element, inserting a guidewire through the catheter, the guidewire comprising at least one magnetic element, magnetically coupling the guidewire to the ferromagnetic component of implanted medical device; and applying an external magnetic field to influence the position or orientation of at least one of the catheter, the guidewire, or the implanted medical device.

In some embodiments, the implanted medical device is selected from the group consisting of an inferior vena cava (IVC) filter, a stent, and a prosthetic heart valve.

In some embodiments, the method further comprises retrieving the implanted medical device.

In some embodiments, provided herein are methods for crossing a stricture in a bodily lumen, the method comprising, advancing a first magnetically-tipped guidewire from a first side of the stricture; advancing a second wire comprising a ferromagnetic portion from a second side of the stricture; and magnetically coupling the first magnetically-tipped guidewire and the second wire across the stricture.

In some embodiments, the method further comprises applying an external magnetic field to guide at least one of the first magnetically-tipped guidewire or the second wire.

In some embodiments, provided here are guidewires for medical procedures, comprising an elongate flexible body having a proximal end and a distal end, and a plurality of magnetic elements disposed in series at or near the distal end of the body.

In some embodiments, the distal end comprises an electrically insulated portion and a non-insulated tip.

In some embodiments, provided herein are catheters for medical procedures, comprising an elongate tubular body defining a lumen and having a proximal end and a distal end, a plurality of magnetic elements disposed at or near the distal end, and at least one radiopaque marker. In some embodiments, the elongate tubular body comprises segments of varying flexibility along its length.

In some embodiments, provided herein are external magnetic devices for use in medical procedures, comprising: a housing; a permanent magnet disposed within the housing; a biocompatible outer layer; and one or more alignment indicators on the housing. In some embodiments, devices further comprise an adjustment mechanism configured to alter the strength or direction of the magnetic field generated by the permanent magnet.

In some embodiments, provided herein methods for performing a medical procedure, comprising: inserting a catheter into a subject, the catheter comprising at least one magnetic element; advancing a guidewire through the catheter, the guidewire comprising at least one magnetic element; generating an external magnetic field; and manipulating at least one of the guidewire or the catheter based on magnetic interactions between the at least one magnetic element of the guidewire, the at least one magnetic element of the catheter, and the external magnetic field.

### BRIEF DESCRIPTION OF THE DRAWINGS

Before any embodiments are explained in detail, it is to be understood that the invention is not limited in its application to the details of construction and the arrangement of components set forth in the following description or illustrated in the following drawings. The invention is capable of other embodiments and of being practiced or of being carried out in various ways.

FIG. 1A. Drawing of an exemplary magnetically-tipped guidewire medical device, including an exposed magnetic tip (1), a series of magnetic elements (2), and non-magnetic portion of the guidewire (3) in a straight configuration.

FIG. 1B. Drawing of the exemplary magnetically-tipped guidewire medical device of FIG. 1A, including an exposed magnetic tip (1), a series of magnetic elements (2), and non-magnetic portion of the guidewire (3) in an angled configuration.

FIG. 2. Drawing of an exemplary external magnet medical device, including a magnetic element (1), a junction between magnetic portion and non-magnetic portion (2), and a non-magnetic portion (3).

### DETAILED DESCRIPTION

Reference will now be made in detail to various embodiments of the subject matter, examples of which are illustrated in the accompanying drawings. While various embodiments are discussed herein, it will be understood that they are not intended to limit to these embodiments. On the contrary, the presented embodiments are intended to cover alternatives, modifications, and equivalents, which may be included in the spirit and scope of the various embodiments.

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “an” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

The term “coupled,” as used herein, is defined as “connected,” although not necessarily directly, and not necessarily mechanically. The term coupled is to be understood to mean physically, magnetically, chemically, fluidly, electrically, optically, or otherwise coupled, connected or linked and does not exclude the presence of intermediate elements between the coupled elements absent specific contrary language. In contrast, when an element is referred to as being “directly on,” “directly engaged to,” “directly connected to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.).

Provided herein are medical devices with magnetic components that allow for locating, repositioning, and/or retrieving medical devices within a subject. In particular, a magnetically tipped wire, a magnetic sheath/catheter, and/or an external magnet are provided, for example, as components of system with other medical devices and/or for use in medical procedures, such as in the field of interventional radiology (IR).

### Magnetically Tipped Wire

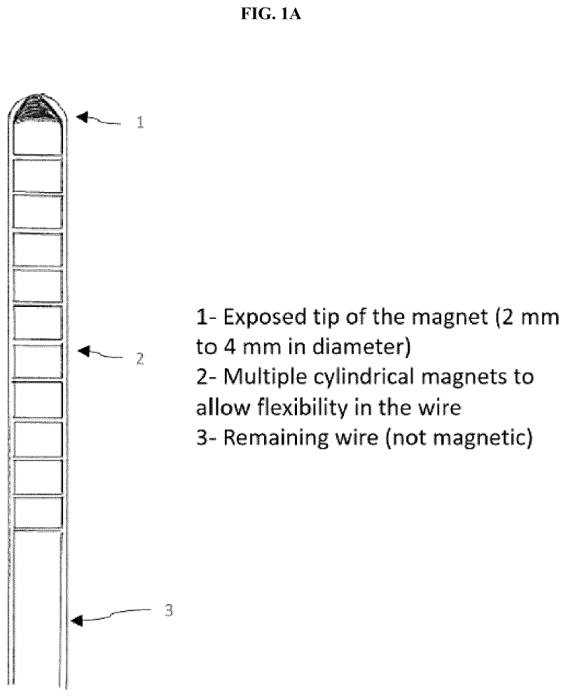

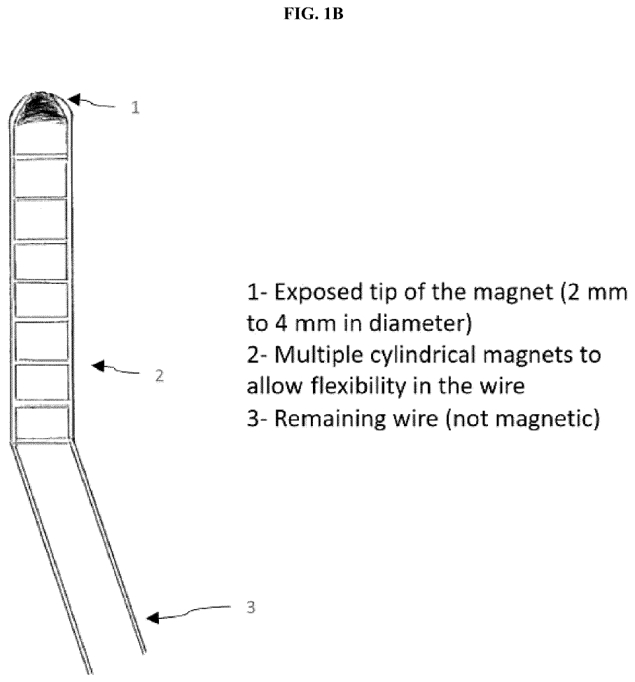

FIG. 1A is a drawing of an exemplary magnetically-tipped guidewire medical device, including an exposed magnetic tip (1), a series of magnetic elements (2), and non-magnetic portion of the guidewire (3) in a straight configuration. FIG. 1B is a drawing of the magnetically-tipped guidewire medical device of FIG. 1A, but in an angled configuration.

In some embodiments, provided herein is a magnetically tipped wire (or “magnetic wire”) for use in various medical procedures. In some embodiments, the magnetic wire finds use, for example, in engaging ferromagnetic components on implanted devices such as inferior vena cava (IVC) filters or stents. In some embodiments, a magnetically tipped wire is used to cross strictures in blood vessels, bile ducts, or other bodily lumens. In some embodiments, the magnetic wire is used to approximate two wires across a stricture. Embodiments herein are not limited by the type of procedure or the locations where the magnetic wire may be used.

In some embodiments, the body of the magnetic wire comprises a thin and flexible material that allows it to navigate through tortuous vasculature or other bodily passages. In some embodiments, the body of the magnetic wire is sufficiently rigid to advance through a catheter or sheath when a force is applied to one end. In some embodiments, the magnetic wire body comprises a flexible, non-magnetic material, such as nitinol (nickel-titanium alloy), mp35n (nickel-cobalt-chromium-molybdenum alloy), phynox (cobalt-chromium-nickel alloy), elgiloy (cobalt-chromium-nickel alloy), titanium, titanium alloys, peck (polyetheretherketone), platinum, platinum-iridium alloys, tantalum, gold, gold alloys, carbon fiber composites, stainless steel (3161-low magnetic permeability austenitic stainless steel), or other suitable alloys. In some embodiments, the magnetic wire body may comprise a combination of materials to optimize flexibility and rigidity.

In some embodiments, the magnetic wire comprises a cylindrical shape with a length of 50-300 cm (e.g., 50 cm, 100 cm, 150 cm, 200 cm, 250 cm, 300 cm, or ranges therebetween) and a diameter of 0.014-0.038 inches (e.g., 0.014 inches, 0.018 inches, 0.025 inches, 0.035 inches, 0.038 inches, or ranges therebetween). In some embodiments, the length and diameter of the magnetic wire are selected based on the specific procedure and anatomical requirements. In some embodiments, the magnetic wire may have a tapered design, with a smaller diameter at the distal end for increased flexibility. In some embodiments, the wire comprises a circular or elliptical cross-section with cross-sectional dimensions between 0.014 and 0.038 inches (e.g., 0.014 inches, 0.018 inches, 0.025 inches, 0.035 inches, 0.038 inches, or ranges therebetween).

A feature of embodiments herein is the inclusion of cylindrical magnets at the tip of the magnetic wire. In some embodiments, the cylindrical magnets are 1-4 mm in diameter (e.g., 1 mm, 2 mm, 3 mm, 4 mm, or ranges therebetween). In some embodiments, multiple cylindrical magnets are aligned in series to increase the magnetic force generated. In some embodiments, the shaft of the distal tip with the magnets is insulated, while the distal tip itself is non-insulated. In some embodiments, the magnets may be encased in a biocompatible material to prevent direct contact with body tissues. Suitable biocompatible materials may include, but are not limited to silicone, polyurethane, ptfe (polytetrafluoroethylene), peek (polyetheretherketone), pe (polyethylene), pp (polypropylene), parylene, epoxy resins (biocompatible grades), polyimide, hydrogel coatings, polycarbonate, stainless steel (3161), titanium, nitinol, ceramic (alumina or zirconia), gold, platinum, etc.

In some embodiments, the magnetic wire comprises a hydrophilic coating to enhance lubricity and reduce friction during navigation through anatomy. In some embodiments, the coating comprises polyvinylpyrrolidone (PVP), hyaluronic acid, or hydrophilic polyurethane. In some embodiments, the magnetic elements are hermetically sealed within a titanium or stainless steel capsule to prevent corrosion and ensure long-term biocompatibility.

In some embodiments, the magnetic attraction between the magnetic wire and a ferromagnetic component on an implanted device is sufficiently strong to cause proper engagement when brought into sufficient proximity (e.g., <3 cm, <2 cm, <1 cm, <0.5 cm, etc.). In some embodiments, the magnetic strength is adjustable or customizable based on the specific application.

In some embodiments, the magnetic elements comprise neodymium-iron-boron (NdFcB) magnets. In some embodiments, the magnetic elements comprise samarium-cobalt (SmCo) magnets for enhanced temperature stability. In some embodiments, the magnetic polarity is oriented axially along the length of the wire to maximize engagement with ferromagnetic targets. In some embodiments, the magnetic elements are arranged with alternating polarities to create a focused magnetic field pattern.

### Magnetic Sheath/Catheter

In some embodiments, provided herein is a magnetic sheath/catheter for use in various medical procedures. In some embodiments, the catheter comprises an outer sheath with cylindrical magnets incorporated into its design. In some embodiments, both the outer sheath and an inner catheter (if present) are elongated tubes (cylinders) with inner lumens. In some embodiments, the magnetic catheter may be designed with varying degrees of flexibility along its length to facilitate navigation through complex anatomical structures. Varying flexibility may be achieved through selection of materials (e.g., any of the magnetic wire body materials or biocompatible materials described herein), through the thickness of the catheter walls, and/or through other structural elements (e.g., ribs, supports, etc.).

In some embodiments, the magnetic catheter comprises an outer diameter suitable to traverse a lumen or lumens of a subject from the insertion site to the location of an implanted device. The outer diameter of the magnetic catheter may be from about 3-16 Fr (e.g., 3 Fr, 4 Fr, 5 Fr, 6 Fr, 7 Fr, 8 Fr, 9 Fr, 10 Fr, 11 Fr, 12 Fr, 13 Fr, 14 Fr, 15 Fr, 16 Fr, or ranges therebetween). In some embodiments, the magnetic catheter may have a variable outer diameter along its length.

In some embodiments, the magnetic catheter comprises a distal end for insertion through a lumen of a subject (e.g., blood vessel, bile duct, ureter, etc.) and a proximal end for manipulation/control by an operator (e.g., clinician). In some embodiments, the proximal end of the magnetic catheter is maneuvered through lumen(s) within a subject to place the distal end in proximity (e.g., <5 cm, <4 cm, <3 cm, <2 cm, <1 cm) with an implanted device. In some embodiments, the magnetic catheter may include radiopaque markers spaced at 1-5 cm intervals (e.g., 1 cm, 2 cm, 3 cm, 4 cm, 5 cm) to aid in positioning under fluoroscopic guidance.

In some embodiments, the magnetic catheter comprises multiple lumens to accommodate simultaneous passage of multiple devices. In some embodiments, a primary lumen accommodates guidewires while secondary lumens accommodate contrast injection, aspiration, or additional guidewires. In some embodiments, the lumens are arranged concentrically or in a side-by-side configuration. In some embodiments, the catheter comprises a port to facilitate guidewire exchanges.

A feature of embodiments herein is the inclusion of cylindrical magnets at the tip of the magnetic catheter. In some embodiments, the cylindrical magnets are 1-3 mm in diameter (e.g., 1 mm, 2 mm, 3 mm, or ranges therebetween). In some embodiments, the magnetic catheter allows passage of wires or other devices through its lumen parallel to the cylindrical magnets. In some embodiments, the magnetic elements may be arranged in various configurations to optimize magnetic coupling with target devices.

In some embodiments, the magnetic catheter comprises electromagnetic shielding along portions of its length to prevent unintended magnetic interactions with surrounding tissues or medical devices. In some embodiments, the shielding comprises mu-metal, permalloy, or other high-permeability magnetic shielding materials. In some embodiments, the magnetic elements are recessed within the catheter wall to prevent direct tissue contact while maintaining effective magnetic coupling. In some embodiments, the catheter comprises a mechanism for selectively activating or deactivating the magnetic field, such as a rotating sleeve that shields or exposes the magnetic elements.

Further disclosure of magnetically-couplable stents and catheters, as well as associated guidewires, retrieval elements, and other components and instruments may be found in PCT Pub. No. WO 2024/073427 (Intl. App. No. PCT/2023/075139; incorporated by reference in its entirety).

### External Magnet

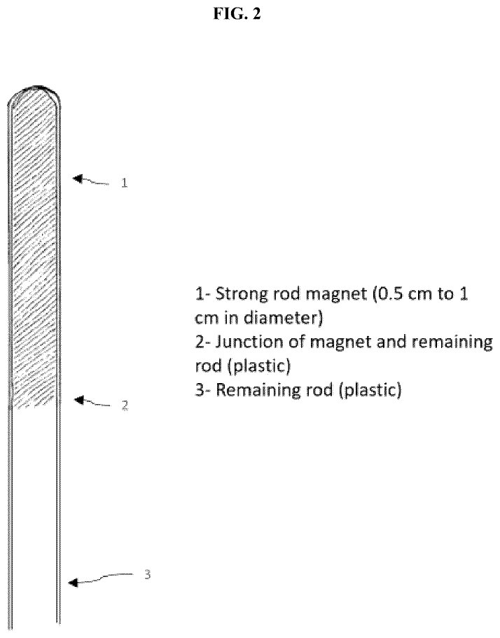

FIG. 2 is a drawing of an exemplary external magnet medical device, including a magnetic element (1), a junction between magnetic portion and non-magnetic portion (2), and anon-magnetic portion (3).

In some embodiments, provided herein is a magnet for external use in various medical procedures. In some embodiments, the magnet is a strong external magnet, for example, with a rod shape. In some embodiments, the magnet finds use in providing strong magnetic forces to internal implanted devices such as magnet or IVC filters to align components for ease of retrieval. In some embodiments, the magnet may be used to attract or repel magnetic components inside the body.

In some embodiments, the magnet comprises a permanent magnet material, such as neodymium, samarium-cobalt, or other rare-earth magnets. In some embodiments, the magnet is coated with a biocompatible material to ensure safety during use. Suitable biocompatible materials include, for example, ilicone, polyurethane, ptfe (polytetrafluoroethylene), peck (polyetheretherketone), pe (polyethylene), pp (polypropylene), parylene, epoxy resins (biocompatible grades), polyimide, hydrogel coatings, polycarbonate, stainless steel (3161), titanium, nitinol, ceramic (alumina or zirconia), gold, platinum, etc. In some embodiments, the magnetic strength of the magnet may be adjustable to suit different clinical scenarios.

In some embodiments, the magnet comprises a rod shape with a length of 10-50 cm (e.g., 10 cm, 20 cm, 30 cm, 40 cm, 50 cm, or ranges therebetween) and a diameter of 1-5 cm (e.g., 1 cm, 2 cm, 3 cm, 4 cm, 5 cm, or ranges therebetween). In some embodiments, the rod shape is selected to minimize obstruction of the visual field during fluoroscopic imaging. In some embodiments, the magnet may have an ergonomic design to facilitate handling and manipulation by the operator.

In some embodiments, the magnetic force generated by the magnet is sufficiently strong to move ferromagnetic components within implanted devices when the magnet is placed on the external surface of a subject's body. In some embodiments, the magnet may include features to aid in precise positioning, such as alignment markers or a targeting mechanism.

### System Integration and Applications

In some embodiments, the systems herein comprise a magnetic wire, a magnetic catheter/sheath, and an external magnet used in combination to perform various medical procedures. In some embodiments, the magnetic wire is inserted through the catheter/sheath to engage an implanted device. In some embodiments, the external magnet is used externally to guide the internal components. In some embodiments, the system may include additional components such as guidewires, retrieval elements, or imaging devices to enhance functionality.

The full scope of embodiments herein is not limited by the type of medical device to be retrieved, the procedure, or the locations where the magnetic wire may be used. However, exemplary medical devices include inferior vena cava (IVC) filters, temporary biliary stents, nephrostomy tubes, percutaneous gastrostomy tubes (PEG or PERC), temporary embolic agents (e.g., detachable coils), esophageal, duodenal, and colonic self-expanding metal stents (SEMS), biliary stents (plastic or metal), pancreatic duct stents, enteral feeding tubes (e.g., PEG, PEJ), Over-the-Scope Clip (OTSC) systems, ureteral stents (double J or JJ stents), temporary prostatic stents, percutaneous access sheaths, etc. In some embodiments, any of these devices, or similar devices may comprise a metal or magnetic element that can be engaged by the magnetic wire.

In some embodiments, the devices and methods herein find use in retrieving IVC filters. In some embodiments, a magnetic wire is advanced through a catheter/sheath to engage a ferromagnetic component on the IVC filter. In some embodiments, the external magnet is used externally to help align the IVC filter for easier engagement with the magnetic wire. In some embodiments, the system may include specialized retrieval mechanisms designed specifically for IVC filter removal.

In some embodiments, the devices and methods herein find use in crossing complex strictures in blood vessels. In some embodiments, a magnetic wire is inserted from a superior aspect (e.g., internal jugular vein) and a corresponding wire with a ferromagnetic metal or opposite-pole magnetic wire is inserted from an inferior aspect (e.g., femoral vein). In some embodiments, the two wires approximate and engage across the narrowing, ensuring safe and effective crossing of the stricture. In some embodiments, the system may include additional tools for treating the stricture once crossed, such as balloon catheters or stent delivery systems.

In some embodiments, the devices and methods herein find use in retrieving plastic stents, such as those used in the biliary or urinary system. In some embodiments, a magnetic wire is used to engage a ferromagnetic component on the stent, while the external magnet is used externally to help guide the stent into a position favorable for retrieval. In some embodiments, the system may include specialized stent retrieval tools that work in conjunction with the magnetic components.

In some embodiments, the devices and methods herein are performed by a human operator. In some embodiments, one or more (e.g., all) of the steps of a method described herein are automated (e.g., performed by a robotic instrument or system). In some embodiments, the system may include sensors and feedback mechanisms to provide real-time information to the operator or robotic system during the procedure.

The magnetic devices described herein offer several advantages over traditional methods. In some embodiments, these devices improve alignment precision, reducing guesswork and the level of skill needed for complex interventions. In some embodiments, the magnetic coupling allows for easier engagement of implanted devices, potentially reducing procedure times and improving patient outcomes. In some embodiments, the ability to manipulate internal components using external magnetic fields provides a non-invasive means of assisting with interventional procedures. In some embodiments, the system may reduce radiation exposure to both patients and operators by potentially decreasing fluoroscopy time required for device manipulation.

In some embodiments, the devices and methods described herein may be adapted for use in other medical fields, such as neurology, cardiology, or orthopedics, expanding their potential applications beyond the initially described interventional radiology, gastroenterology, and urology procedures.

What is claimed is:

1. A medical device system comprising:
the guidewire of claim 14;
a catheter comprising a lumen configured to receive the guidewire and at least one magnetic element disposed at a distal portion of the catheter; and
an external magnetic device configured to generate a magnetic field detectable within a body of a subject.

2. The system of claim 1, wherein the at least one magnetic element of the guidewire comprises a plurality of cylindrical magnets aligned in series.

3. The system of claim 1, wherein the guidewire comprises a non-magnetic material selected from the group consisting of nitinol, stainless steel, and combinations thereof.

4. The system of claim 1, wherein the guidewire has a length between 50 cm and 300 cm and a diameter between 0.014 inches and 0.038 inches.

5. The system of claim 1, wherein the at least one magnetic element of the catheter comprises a plurality of cylindrical magnets.

6. The system of claim 1, wherein the catheter has an outer diameter between 3 Fr and 16 Fr.

7. The system of claim 1, wherein the external magnetic device comprises a rod-shaped permanent magnet having a length between 10 cm and 50 cm and a diameter between 1 cm and 5 cm.

8. The system of claim 1, further comprising an implantable medical device having a ferromagnetic component configured to interact with the at least one magnetic element of the guidewire.

9. A method for manipulating an implanted medical device, the method comprising:
advancing a catheter through a lumen of a subject to a location proximate to the implanted medical device, the catheter comprising at least one magnetic element;
inserting the guidewire of claim 14 through the catheter;
magnetically coupling the guidewire to the implanted medical device; and
applying an external magnetic field to influence the position or orientation of at least one of the catheter, the guidewire, or the implanted medical device.

10. The method of claim 9, wherein the implanted medical device is selected from the group consisting of an inferior vena cava (IVC) filter, a stent, and a prosthetic heart valve.

11. The method of claim 9, further comprising retrieving the implanted medical device.

12. A method for crossing a stricture in a bodily lumen, the method comprising:
advancing a first magnetically-tipped guidewire from a first side of the stricture;
advancing a second wire comprising a ferromagnetic portion from a second side of the stricture; and
magnetically coupling the first magnetically-tipped guidewire and the second wire across the stricture.

13. The method of claim 12, further comprising applying an external magnetic field to guide at least one of the first magnetically-tipped guidewire or the second wire.

14. A guidewire for medical procedures, comprising:
an elongate flexible body having a proximal end and a distal end; and
at least one magnetic element disposed at the distal end of the body.

15. The guidewire of claim 14, wherein the distal end comprises an electrically insulated portion and a non-insulated tip.

16. A sheath/catheter for medical procedures, comprising:
an elongate tubular body defining a lumen and having a proximal end and a distal end;
a plurality of magnetic elements disposed at or near the distal end; and
at least one radiopaque marker.

17. The catheter of claim 16, wherein the elongate tubular body comprises segments of varying flexibility along its length.

18-19. (canceled)

20. A method for performing a medical procedure, comprising:
inserting a catheter into a subject, the catheter comprising at least one magnetic element;
advancing the guidewire of claim 14 through the catheter;
generating an external magnetic field; and
manipulating at least one of the guidewire or the catheter based on magnetic interactions between the at least one magnetic element of the guidewire, the at least one magnetic element of the catheter, and the external magnetic field.

21. The guidewire of claim 14, wherein the at least one magnetic element disposed at the distal end of the body comprises a plurality of magnetic elements disposed in series at or near the distal end of the body.
